(19) United States
(12) Patent Application Publication
(10) Pub. No.: US 2009/0301176 A1
(43) Pub. Date: Dec. 10, 2009

(54) Microelectromechanical System Comprising a Deformable Portion and a Stress Sensor

(75) Inventors: Alistair Rowe, Orsay (FR); Christoph Renner, London (GB); Steve Arscott, Lambersart (FR)

Correspondence Address: MILLER, MATTHIAS & HULL, ONE NORTH FRANKLIN STREET, SUITE 2350, CHICAGO, IL, 60606, US

(73) Assignee: ECOLE POLYTECHNIQUE, Palaiseau Cédex (FR)

(21) Appl. No.: 12/096,314

(22) PCT Filed: Dec. 12, 2006
(86) PCT No.: PCT/FR2006/002713

§ 371 Date: May 28, 2009

(30) Foreign Application Priority Data
Dec. 15, 2005 (FR) 0512740

(51) Int. Cl. G01N 29/00; H01L 41/00; G12B 21/08; G01N 33/483; B81B 3/00

(52) U.S. Cl. 73/64.53; 310/314; 850/33; 73/53.01

(57) ABSTRACT

A microelectromechanical system comprises a deformable portion and at least one stress sensor fixedly attached to the deformable portion. The sensor itself comprises a base portion and a shunt portion juxtaposed on the deformable portion, and connections arranged to detect a change of a distribution of an electric current in the base and shunt portions. Such a system is suitable for many applications, in particular for forming a portion of an arm of an atomic force microscope or for entering into the constitution of a bio sensor.

The present invention relates to a microelectromechanical system that comprises a moving part and a stress detector.

A microelectromechanical system or MEMS is an integrated electronic device that includes a moving part. It is produced using standard fabrication techniques for an integrated electronic circuit, including those for producing the moving part. Such systems may form in particular, resonators or microswitches. Some of these comprise a detector for detecting the position of the moving part. This detector makes it possible to monitor a state of the system or to deliver an electrical output signal that corresponds to displacements of the moving part, for example when the system constitutes a frequency filter. Known position detectors consist of a capacitor, the capacitance of which varies as a function of the position of the moving part, or consist of the gate of an MOS transistor carried on the moving part. The operation of these position detectors depends on the dielectric conductivity of the ambient medium present around the moving part. Now, for some applications, in particular in a liquid or gaseous medium, this conductivity may undergo fluctuations that generate imprecision in the results of the detection. Other detectors incorporated into MEMS consist of a portion of a piezoelectric material. However, in this case the position of the moving part of the MEMS is known only for an alternating stress.

High-resolution detectors for detecting the position of a moving part have moreover been developed that do not have some of the above drawbacks. Among such detectors, mention may be made of optical detectors that are based on the reflection of a light beam, in general laser beam, on the moving part. A point of impact of the beam on a set of photodetectors then varies as a function of the position of the moving part. However, such optical detectors have other drawbacks: they are bulky and require the moving part to be reflective and the ambient material present around said part to be transparent. Furthermore, precise optical alignment and calibration are essential. Finally, the use of a light beam is not compatible with certain applications, especially chemical applications, since the beam may induce parasitic photochemical reactions liable to impair the interpretation of the measurement.

There are also detectors that are based on a tunnel effect present between two portions of conductive materials that are very close to each other. In these detectors, the intensity of a tunnel effect electrical current is used to measure a distance between the two portions. However, the characteristic of such a detector is highly nonlinear and is incompatible with the elastic constants of the moving parts currently produced within the MEMS. Furthermore, it is necessary for such a tunnel type detector to be used with a high vacuum condition so that the tunnel current is sufficiently stable to constitute a representative detection signal. This results in considerable constraints having an impact on the environment, which limit the use of a detector of this type.

Finally, in certain MEMS, the detector for detecting the position of the moving part is replaced by a stress detector. Such a detector is generally based on the piezoresistive behavior of the moving part of the MEMS so that it is less sensitive to external perturbations than a position detector. However, the sensitivity of the detection is generally low, as it is limited by the intrinsic piezoresistance of the constituent material of the moving part.

One object of the present invention therefore consists in proposing a microelectromechanical system that incorporates a detector for detecting a state of the system but that does not have the abovementioned drawbacks.

Another object of the present invention consists in proposing a microelectromechanical system that can be used for many applications, and in particular that can be used in a variety of ambient media.

To do this, the invention provides a microelectromechanical system that comprises a deformable part and at least one stress detector firmly joined to the latter. Each stress detector itself comprises:
a base portion and a shunt portion which are electrically conducting and juxtaposed on the deformable part in such a way that the base portion and the shunt portion are in electrical contact with each other along respective adjoining sides of said portions, the shunt portion having an electrical conductivity higher than the conductivity of the base portion; and
a set of electrical connections that are connected to the base portion, away from a contact zone where said base portion is in contact with the shunt portion, and are arranged so that a modification in the distribution of an electrical current in the base and shunt portions may be detected electrically using said connections.

Furthermore, respective materials of the base and shunt portions are chosen so that a contact resistance between said portions varies as a function of a deformation of the system.

Thus, according to the invention, a deformable part and a detector are combined in one and the same microelectromechanical system. Such a combination makes it possible to obtain a compact assembly. The system can then be easily introduced into many apparatus corresponding to different applications.

Furthermore, this combination of the deformable part and the detector in one and the same system makes it possible to use a coherent fabrication process for producing both the deformable part and the detector. This results in a reduction in the overall fabrication cost. In particular, the system can be entirely produced using the techniques for fabricating integrated electronic circuits, which techniques are thoroughly understood at the present time and make it possible to miniaturize the system and to carry out mass production with high fabrication yields. These fabrication techniques also allow an electronic measurement circuit to be integrated into the microelectromechanical system so as to obtain high sensitivity and high measurement precision.

The detector incorporated into the microelectromechanical system is of the stress detector type. Such a detector delivers particularly reliable detection results. In particular, given that a detector of this type is directly attached to a part intended to undergo deformations, the detection results are not disturbed by variations in the dielectric conductivity of a fluid medium in contact with the system. A system according to the invention can therefore be used for a large number of applications, especially applications for which the system is immersed in a fluid, whether liquid or gaseous.

Moreover, the sensitivity of the stress detector used in a system according to the invention is based not only on variations in the contact resistance between the base portion and the shunt portion, but also results from a difference between the electrical conduction properties of the respective materials of the two portions. The sensitivity obtained is high and allows very low deformations of the microelectromechanical system to be detected.

Preferably, the base portion is made of a semiconductor material and the shunt portion is of metallic type. The base and shunt portions then have a large difference in electrical conductivity, and the sensitivity of the detector is then even higher. Under these conditions, deformations that correspond to displacements of the order of 1 \dot{a}ngström of the moving part may be detected. As examples, the base portion may be made of silicon (Si), on an indium alloy (In) and on antimony (Sb) or a gallium (Ga) and arsenic (As) alloy, and the shunt portion may be made of aluminum (Al) or of gold (Au), especially.

More preferably, the material of the base portion may have a variable electrical conductivity, the anisotropy of which changes when this material is subjected to a stress. Such an anisotropy appears when the stress is applied to the base portion and may be oriented according to the direction of the stress. Furthermore, two conductivity values of the material of the base portion, which correspond to different directions, have a difference between them that depends on the amplitude of the stress, and the sign of this difference may depend on the nature of the stress, whether a compressive or tensile stress, and also on the type of electrical doping of the material of the base portion.

Such a variable anisotropy of the electrical conductivity of the material of the base portion may appear when this material is an amorphous, polycrystalline or single-crystal material. For example, the base portion may be based on substantially single-crystal silicon of cubic structure. In this case, a particularly high detection sensitivity is obtained when the [110] axis of this cubic structure is approximately perpendicular to the respective sides of the mutually adjoining base and shunt portions.

To further increase the sensitivity of the detector, the set of electrical connections may comprise at least three connections, two of which connections are designed to supply the detector with electrical current and two connections are designed to detect a voltage created by this current. Such a configuration makes it possible to reduce or eliminate a contribution to the measured voltage between the detection connections that would be generated by contact resistances of connections. Optionally, one of the connections may be used both for supplying current and for detecting the voltage.

Alternatively, the set of electrical connections of the detector may comprise only two connections, each of which is used both for supplying current and for detecting the voltage. In this case, an improved ratio of the detected voltage to a detection noise is obtained.

The stress detector is preferably placed on the deformable part in a zone of the latter in which the deformations and/or stresses are large or maximal. More precisely, the interface between the base and shunt portions, or a central part of the base portion, is located in that zone of the deformable part where the deformations and/or the stresses are maximal. Such positioning of the detector depends on the physical parameter, which varies most as a function of the stresses transmitted to the detector: the conductivity anisotropy of the material of the base portion or the electrical contact resistance between the base and shunt portions.

According to a preferred configuration of the microelectromechanical system, the base and shunt portions may be integrated into the deformable part without protruding from a surface of this part. In this case, the presence of the stress detector on the surface of the deformable part does not modify the mechanical properties thereof. In particular, the elasticity, the mass and the eigenfrequency of the mechanical oscillation of the deformable part are not modified by the presence of the detector, or are only very slightly modified.

Optionally, the microelectromechanical system may comprise two stress detectors, which are firmly joined to the deformable part. According to one particular configuration of the system, the two detectors are placed on two opposed faces of the deformable part. In this case, they may be placed so as to provide a differential measurement of a deformation. The expression “differential measurement of a deformation” is understood, within the context of the present invention, to mean a measurement that results from a subtraction operation between detection signals delivered by two respective stress detectors placed on the deformable part. For such a differential measurement, the system may further include at least one electronic differential measurement circuit connected to connections that connect the two respective detectors. This circuit may also be integrated into the microelectromechanical system. For example, such a differential measurement circuit may comprise a bridge, of the Wheatstone bridge or Kelvin bridge type, or any other differential measurement bridge, of single of double structure, known to those skilled in the art.

According to a preferred embodiment of the invention, the deformable part of the system may comprise a beam. Such a beam may be deformed in various ways, especially by flexure perpendicular to the longitudinal direction of the beam, by torsion or by elongation-compression. At least one stress detector is preferably placed on the beam at a point where the deformations of the surface of the latter are particularly high. Such a point may be located in particular at mid-length of the beam. Advantageously, the beam may have a free end and an end that is rigidly connected to a fixed part of the system. The deformations of the beam then correspond to displacements of the free end of the beam. For such a configuration of the deformable part, and depending on the uses of the microelectromechanical system, at least one stress detector may preferably be located close to the end of the beam that is connected to the fixed part.

In particular, the electrical connections may be provided on the base portion of each detector so that a deformation of the beam, by flexure, by torsion or by elongation-compression, can be detected electrically by means of these connections. Optionally, they may be arranged in such a way that deformations of the beam in two different modes can be detected separately. In particular, deformations of the beam by flexure in one direction and by torsion, or else flexural deformations in two perpendicular directions, may be detected separately and simultaneously by means of the electrical connections.

Such a microelectromechanical system is suitable for many applications. The invention also provides an atomic force microscope, an analyzer for analyzing the composition of a fluid, a biodetector, a biological analyzer, an accelerometer and a sensor for detecting a property of a fluid, each of the above comprising at least one microelectromechanical system according to the invention.

The invention also proposes to use such a microelectromechanical system to detect a deformation of a support to which the system is attached. Such a support may for example be part of a mechanical structure liable to deform or a separation membrane between two compartments having the respective internal pressures that are liable to vary one with respect to the other.

Finally, the invention provides a process for producing such a microelectromechanical system as described above. The system is produced from a substrate that comprises a main part with an upper layer, the upper layer being separated from the main part of the substrate by an intermediate layer. The process comprises the following steps:
/a/ a conducting portion made of a first electrically conductive material is formed on the surface of the upper layer, said conducting portion comprising at least the base portion;
/b/ the shunt portion made of a second material, which is more conducting than said first material, is formed on the surface of the upper layer and against the base portion;
/c/ a set of electrical connections connecting the base portion away from a contact zone between the base portion and the shunt portion is formed;
/d/ the upper layer is etched on at least two opposed sides of a residual part of said upper layer, said residual part extending from a zone of the upper layer bearing the base and shunt portions; and
/e/ the main part of the substrate and the intermediate layer are removed in a zone of the substrate containing the residual part of the upper layer.

The residual part of the upper layer then forms the deformable part of the microelectromechanical system.

The main part of the substrate and the upper layer may each be made of a semiconductor material, such as a silicon (Si)-based material. The intermediate layer may be an electrically insulating material, such as silica (SiO₂). The process is then particularly inexpensive, especially because it can be carried out starting from a commercially available substrate of the SOI (silicon on insulator) type. In addition, it may be implemented using some of the production tools that are commonly used to fabricate integrated electronic circuits. In particular, a lithographic process may be used for forming at least one of the two, base and shunt, parts. Optionally, the upper layer may be substantially single-crystal, polycrystalline or amorphous layer.

Preferably, the conducting portion made of the first conductive material may be formed in step /a/ by a first doping of part of the upper layer.

One particular method of implementing such a process makes it possible to obtain a microelectromechanical system in which the base and shunt portions are integrated into the deformable part of the system, without protruding from a surface of this part. In this case, step /b/ may comprise the following substeps:
/b1/ a second doping of the upper layer is carried out selectively in part of said upper layer corresponding to the shunt portion, said second doping having a higher concentration than the concentration of the first doping;
/b2/a portion of a metallic material is deposited on the upper layer, selectively on top of the shunt portion; and
/b3/the substrate is heated so that the metallic material diffuses into the upper layer, penetrating into the shunt portion.

Optionally, the second doping is carried out in step /b1/ in part of the conducting portion, which is produced by the first doping in step /a/, adjacent to the base portion. An interface between the base and shunt portions is then obtained, which possesses electrical and mechanical characteristics highly favorable as regards the lifetime and the sensitivity of the stress detector.

Optionally, the electrical connections may also be formed without protruding from the deformable part, at the same time as the shunt portion. To do this, steps /b/ and /c/ are performed simultaneously and in which:
the conducting part produced by the first doping of step /a/ in the upper layer corresponds to the joining of the base portion, the shunt portion and the electrical connections;
the second doping of step /b1/ is carried out simultaneously and selectively in parts of the conducting portion corresponding to the shunt portion and to the electrical connections;
portions of metallic material are deposited in step /b2/simultaneously and selectively on top of the shunt portion and of the electrical connections; and
the heating of step /b3/is performed so that the metallic material diffuses simultaneously into the shunt portion and into the electrical connections.

Other features and advantages of the present invention will become apparent in the following description of the nonlimiting exemplary embodiments and methods of implementation, with reference to the appended drawings in which:

FIGS. 1a-1c are perspective views of three microelectromechanical systems according to the invention;

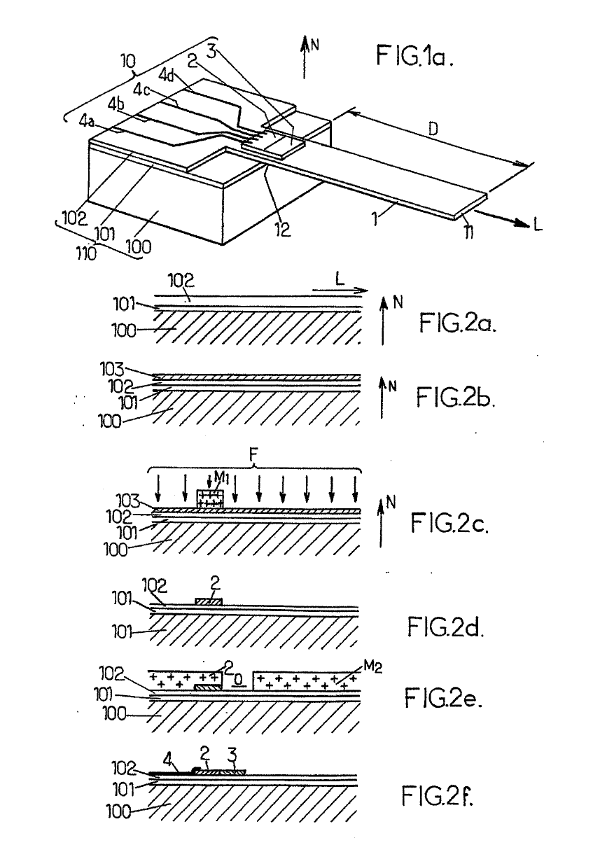

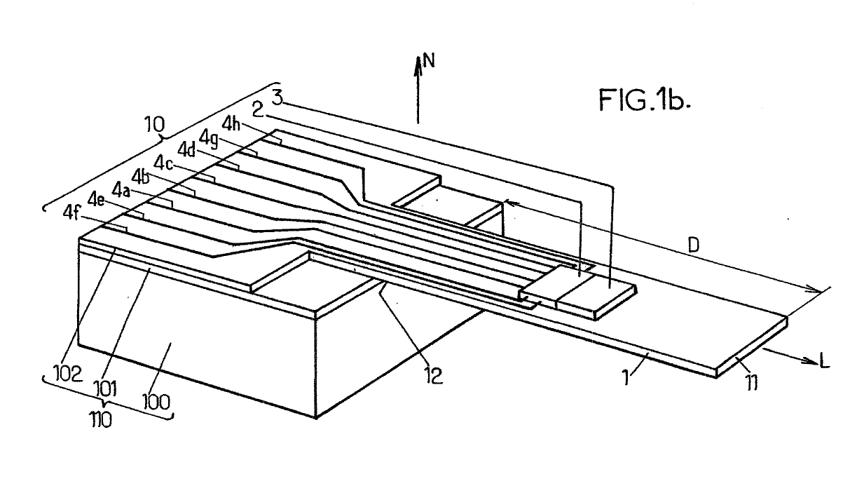

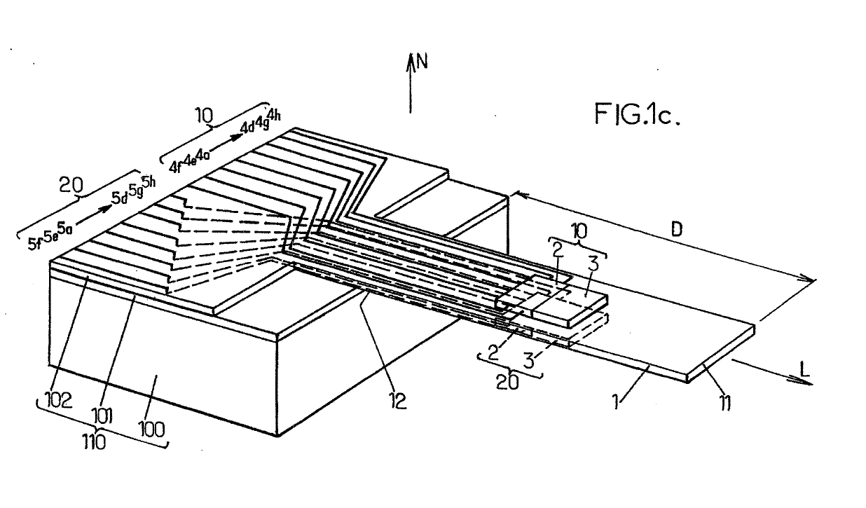

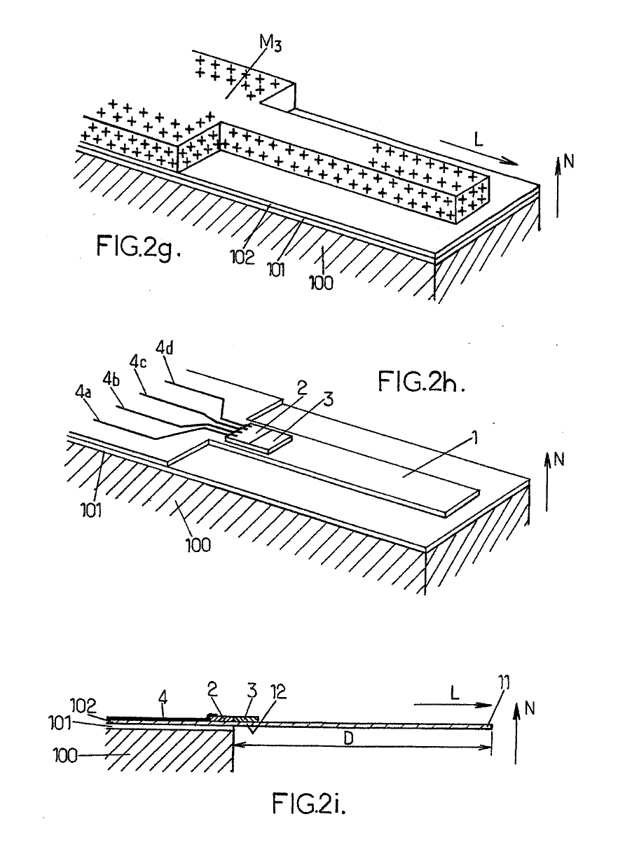

FIGS. 2a to 2i illustrate successive steps of a first process for producing a system according to FIG. 1a;

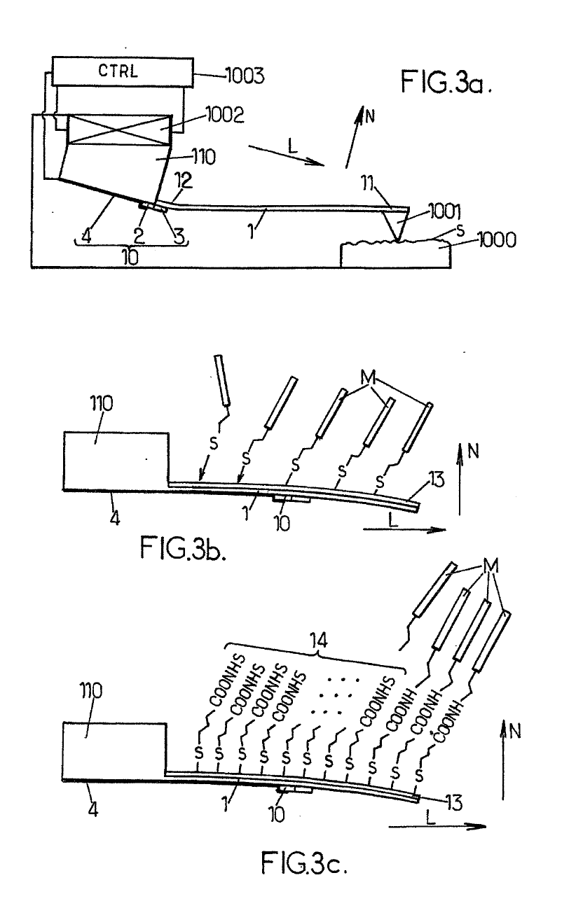

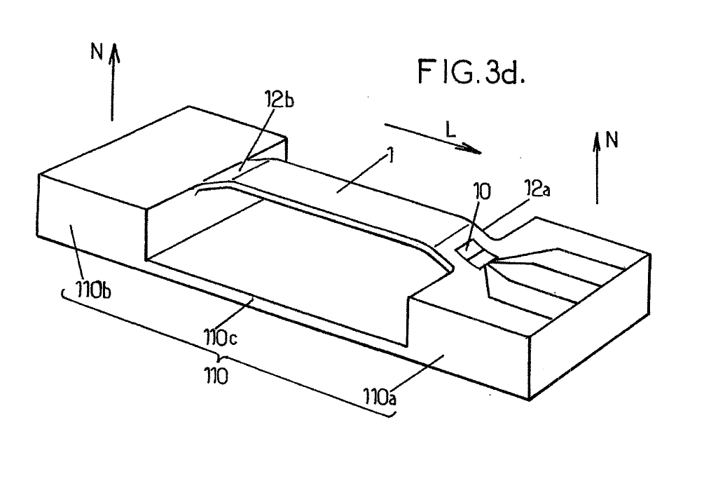

FIGS. 3a-3d illustrate various uses of microelectromechanical systems according to the invention;

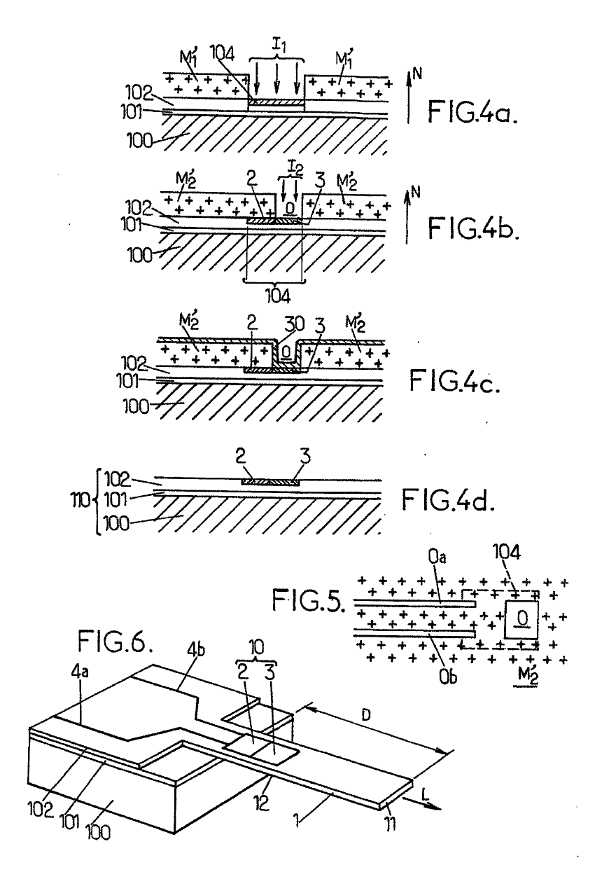

FIGS. 4a-4d illustrate successive steps of a second process for producing a microelectromechanical system according to the invention;

FIG. 5 illustrates an improvement of the second process shown in FIGS. 4a-4d; and

FIG. 6 is a perspective view of a microelectromechanical system obtained by the second process shown in FIGS. 4a-4d and 5.

In these figures, identical references denote identical elements or those that have identical functions. Furthermore, the dimensions of the microelectromechanical system parts shown are not in proportion with actual dimensions or ratios of dimensions. In particular, for the sake of clarity of the figures, dimensions along different directions are not necessarily reproduced with the same scale factor. In the figures, N and L denote an upwardly oriented direction and a longitudinal direction, respectively, of each microelectromechanical system represented.

In accordance with FIGS. 1a-1c, a microelectromechanical system comprises a beam 1, a fixed part 110 and at least one stress detector 10. The beam 1 extends parallel to the direction L over a length D starting from one side of the fixed part 110. The beam 1 may for example be a plate with a thickness of about 1 μm (micron) along the direction N, of length D equal to around 100 microns approximately and of width equal to around 10 microns approximately. It may deform by flexure in a plane parallel to the directions N and L and optionally by torsion about an axis parallel to the direction L. Preferably, the beam 1 consists of a substantially single-crystal or amorphous material so as to have a high elasticity without undergoing irreversible plastic deformations. In this way, the microelectromechanical system has a maximal lifetime. To achieve this, the beam 1 may in particular be made of single-crystal or amorphous silicon.

The fixed part 110 comprises a main part 100, an intermediate layer 101 of silica (SiO₂) and an upper layer 102 of silicon. In the particular embodiments described, the beam 1 consists of an extension of the layer 102 beyond the side of the fixed part 110.

The detector 10 comprises a base portion 2 and a shunt portion 3. The base portion 2 may be made of an n-doped or p-doped semiconductor with an electrical charge carrier density between 10¹⁴ cm⁻³ and 10²⁰ cm⁻³. The shunt portion 3 may be metallic, for example made of gold. The portions 2 and 3 are for example rectangular and in contact with each other along adjoining respective faces. The detector 10 is located on the system in such a way that the faces of the portions 2 and 3, along which these portions are in contact with each other, are parallel to that side of the fixed part 110 from which the beam 1 extends.

In the first exemplary embodiment illustrated by FIG. 1a, the stress detector 10 is located astride the beam 1 and the fixed part 110, approximately level with the point where the beam 1 joins the part 110. The contact faces of the portions 2 and 3 may furthermore be located plumb with that side of the part 110 from which the beam 1 extends. In this way, the stress detector is located at a point on the system where the stresses are particularly high, when these stresses result in a force exerted on the free end 11 of the beam 1 for example. Alternatively, the portion 2 may be itself located astride the beam 1 and the fixed part 110. In both these circumstances, a high sensitivity of the detector 10 relative to movements of the beam 1 is obtained.

A series of electrical connections is placed on the system. These connections are in electrical contact with the base portion 2 and bear the references 4a, 4b, 4c and 4d respectively. They may be arranged so as to form a four-electrode electrical voltage detector. A DC or AC electrical current can then be delivered into the detector 10 via two connections, for example 4a and 4d, so as to supply the detector. An electrical voltage created by this current is detected between two other connections, for example 4b and 4c.

Alternatively, only two electrical connections may be used to form a two-electrode electrical voltage detector. These two connectors are in electrical contact with the base portion 2. Each of them then is used both for delivering the DC or AC electrical current into the detector 10 and for detecting the electrical voltage created by this current.

FIG. 1b illustrates a second embodiment of a microelectromechanical system according to the invention in which the detector 10 is located midway along the beam 1. Furthermore, the system comprises eight electrical connections connecting the base portion 2. The connections 4a-4d may be arranged on the base portion 2 as in the first embodiment illustrated by FIG. 1a. The connections 4e and 4f on the one hand, and the connections 4g and 4h on the other, are located on two opposed lateral sides of the base portion 2. The electrical supply current, whether DC or AC, can then be delivered via the connections 4b and 4h and electrical voltage can be detected between, for example, the connections 4a and 4e. It is also possible to use one and the same connection to deliver the electrical current and to detect the voltage. In this way, the electrical current can be delivered via the connections 4g and 4h and the electrical voltage can be detected using for example the connections 4g and 4c. The connection 4g then has two functions, namely supply and detection. A person skilled in the art will understand that the connections may thus be selected in pairs, to deliver the supply current and to detect the voltage, so as to detect variations in the distribution of the current in the portions 2 and 3, which are caused by flexure, twisting and/or elongation-compression of the beam 1, with a high sensitivity.

FIG. 1c illustrates a third embodiment of a microelectromechanical system according to the invention in which two stress detectors 10 and 20 are placed on and under the beam 1 respectively. Preferably, to obtain a differential detection that provides even higher sensitivity, the two detectors are placed at the same level along the length of the beam 1 so that they deliver amplitude-correlated detection signals. A separate set of connections connects the base portion 2 of each of the two detectors, namely connections 4a-4h for the detector 10 and connections 5a-5h for the detector 20. The connections 5a-5h may pass through the layer 102 through appropriate vias so as to allow easy electrical connection to the upper face of the fixed part 110.

A first process for producing a microelectromechanical system according to FIG. 1a, taken as an example, will now be described. Referring to FIG. 2a, an integrated electronic circuit substrate comprises a main silicon part 100 which is covered on an upper surface with an electrically insulating layer 101 and with a single-crystal or amorphous silicon layer 102. Such a substrate is commercially available and intended for the fabrication of an electronic circuit in SOI technology. For example, the layers 101 and 102 have respective thicknesses of 200 nm and 1000 nm. The layer 102 is made of undoped silicon.

A full-wafer surface doping of the layer 102 is carried out by implantation of dopants with a defined concentration and at a determined depth starting from the upper surface of the layer 102. Such an implantation, which is limited depthwise in the direction N is obtained by scanning the surface of the layer 102 with a beam of dopant particles accelerated by a controlled voltage. A layer 103 of doped silicon, which has a thickness for example of 500 nm, is thus obtained on top of an undoped residual part of the layer 102 (FIG. 2b). Hereafter, the reference 102 consequently denotes the undoped residual part of the upper silicon layer.

A first lithographic resist mask M1 is formed on the layer 103 at the location provided for the base portion 2 (FIG. 2c). The lithographic process used to form the mask M1 is considered to be known, and is not recalled here. Next, the layer 103 is removed outside the mask M1, for example by directing the beam F of accelerated plasma particles against the upper surface of the system, parallel to the direction N but in the opposite sense thereto. Such a method of removal is denoted by the term dry etching. It is continued for a time long enough for the layer 103 to be etched over its entire thickness. The mask M1 is then dissolved in an appropriate solution. A first mesa structure is thus obtained which constitutes the base portion 2 of the detector 10 (FIG. 2d).

A second lithographic resist mask M2 is then formed on the entire layer 102 except for a place intended for the shunt portion 3 (FIG. 2e). In particular, the mask M2 covers the base portion 2 and has an opening O contiguous with one side of the latter. A metallic layer, for example a gold layer, is then deposited on the entire system, for example by thermal evaporation. The mask M2 is then removed (FIG. 2f). A residual part of the metallic layer, in the opening O of the mask M2, forms a second mesa structure that constitutes the shunt portion 3. It is located against the base portion 2. Optionally, a titanium layer (not shown) may be formed on the system before the gold layer so as to increase the adhesion of the shunt portion 3 to the base portion 2.

Electrical connections, indicated overall by the reference 4 in FIG. 2f, of ohmic type, are then formed. The method of forming ohmic contacts of these connections is considered to be known, and is not recalled here.

A lithographic resist mask M3 (FIG. 2g) is then formed on the layer 102. The shape of the mask M3 corresponds to the perimeter of the beam 1 in a plane perpendicular to the direction N. This mask covers in particular the portions 2 and 3 and the connections 4. The layer 102 is then etched in the zones not protected by the mask M3, until the silica layer 101 is exposed. A silicon etching process is advantageously used that is selective relative to the silicon material of the layer 101. The lateral edges and the end of the beam 1 are thus formed. The mask M3 is then dissolved (FIG. 2h).

Finally, a portion of the substrate is removed from under the beam 1, via the lower face of the system, firstly using a process for etching the silicon of the part 100 and then a process for etching the silica of the layer 101. The use of a silica etching process that is selective relative to the silicon material ensures that the beam 1 is not damaged during this step. The system then has the configuration shown in FIGS. 1 and 2i, in which the beam 1 has a fixed end 12, which is rigidly connected to the part 110 of the system, and a free end 11.

The end 11 may undergo displacements in accordance with various deformation modes of the beam 1. Flexure of the beam in a plane parallel to the directions N and L constitutes a first deformation mode. During successive flexures of the beam 1 in the same sense and the direction N and then in the opposite sense, the interface between the base portion 2 and the shunt portion 3 is subjected to a compressive and then a tensile stress. Each of these stresses causes a variation in the electrical resistance of the interface between the portions 2 and 3. When the value of this resistance increases, the current supplied to the detector 10 is distributed more in the base portion 2, and the voltage detected between two of the connections 4a-4h becomes very high. Conversely, when the value of the electrical resistance of the interface between the base portion 2 and the shunt portion 3 decreases, a larger part of the supply current passes by the shunt portion 3, and the voltage detected between the two connections decreases. The two connections 4c and 4b may be used for example to detect a deformation of the beam 1 for flexure in a plane parallel to the directions N and L. The measured electrical voltage is then U_{cb}.

A twisting of the beam 1 about an axis parallel to the direction L and a flexure of the beam within a plane parallel to the layer 102 constitute two other deformation modes of the beam 1. These two other modes are antisymmetric: the two halves of the interface between the portions 2 and 3, on either side of the plane of symmetry of the beam 1 which is parallel to the directions N and L, are subjected to opposite stresses. Deformations of the beam 1 in each of these two other modes may be detected in several ways. In particular, it is possible to calculate the difference U_{ba}-U_{dc}, where U_{ba} is the electrical voltage present between the connections 4b and 4a and U_{dc} is the electrical voltage present between the connections 4d and 4c. Alternatively, when the base portion 2 is connected by eight electrical connections, it is possible to calculate the difference U_{hg}-U_{fe}, where U_{hg} is the electrical voltage present between the connections 4h and 4g and U_{fe} is the electrical voltage present between the connections 4f and 4e. Thus, a torsion of the beam 1 or a flexure in the plane of the layer 102 may be detected independently of and at the same time as a flexure in the direction N, by simultaneously measuring the voltages U_{ba}, U_{cb} and U_{dc} or else U_{hg}, U_{cb} and U_{fe}.

More preferably, the variation in voltage measured during a deformation of the beam 1 in flexure or in torsion may be obtained by selecting, for the base portion 2, a material that has a variable electrical conductivity, the anisotropy of which changes when this material is subjected to a stress. Such an anisotropy appears when the stress is applied to the base portion 2.

When the microelectromechanical system includes a second stress detector placed under the beam 1 (FIG. 1c), the second detector is produced on the lower face of the beam 1 by turning the system over and repeating the steps of forming a stress detector that were described above in relation to FIGS. 2a-2f. Optionally, the thicknesses of the layers 102 and 103 are modified so as to produce the base portions 2 of the two detectors in the initial thickness of the layer 102. Alignment steps are also carried out during production of the lithographic resist masks M1 and M2 for the second detector, for example in order for the two detectors to be located plumb with each other along the direction N.

Such a two-detector system allows a differential, potentially twice as sensitive, measurement of the movements of the moving part. In particular, a flexure of the beam 1, during which the free end 11 moves parallel to the direction N and in the same sense thereof, generates the following stresses:
a compressive stress σ at the upper detector 10 (FIG. 1c), thereby producing a variation ΔU in the voltage detected between two of the connections 4a-4h; and
a tensile stress −σ at the lower detector 20, thereby producing an opposite variation −ΔU in the voltage detected between the two connections of the set 5a-5h that are arranged symmetrically to the above two connections of the set 4a-4h with respect to the beam 1.

Using a known electronic differential detection circuit, for example of the Wheatstone bridge or Kelvin bridge type, and which can be integrated into the layer 102, a voltage variation equal to 2×ΔU can be measured.

A second advantage of a two-detector system configuration, which allows a differential measurement to be performed, lies in the fact that the measurement is insensitive to an elongation-compression of the beam caused, for example, by a temperature variation. This is because the variations in electrical voltages produced in the two detectors by the variation in temperature are pairwise equal and are eliminated in the voltage subtraction operation carried out in the differential detection circuit.

The above microelectromechanical systems are suitable for many applications, especially using the deformation mode of the beam 1 by flexure along the direction N.

In accordance with a first application, the beam 1 forms part of one arm of an atomic force microscope intended for detecting variations in height of an observed surface. As is known, such a microscope comprises a tip located at the free end of an arm and displaced on the observed surface. Variations in at least one structural, physical and/or chemical property of the surface, such as variations in height or in friction, cause deformations of the arm by flexure, which are detected. According to one standard method of operating such a microscope, the surface is observed by moving an opposite end of the beam perpendicular to the observed surface so that the beam undergoes a constant deformation when the tip is moved between points on the observed surface that correspond to different heights. FIG. 3a illustrates schematically the implementation of a microelectromechanical system according to the invention within an atomic force microscope of this type. The free end 11 of the beam 1 is provided with a tip 1001 intended to travel over the surface S of a specimen 1000. The fixed part 110 of the system is mounted on a piezoelectric actuator 1002 designed to move the part 110 parallel to the surface S. The stress detector 10 is located close to the point where the beam 1 joins the part 110, as in FIG. 1a. The displacements parallel to the surface S make it possible to scan the latter. Simultaneously, the detector 10 is supplied with a current between the connections 4a and 4d, these two connections being mentioned by way of example, and displacement along the direction N are controlled by a control unit 1003, denoted by CTRL, so that the voltage detected between two other terminals, for example 4b and 4c, is constant. The displacements along the direction parallel to the surface S that are controlled by the unit 1003 reproduce the variations in height of the surface S and are recorded. The operation of such a microscope is entirely electrical—this is particularly simple and requires neither optical calibration nor alignment. Furthermore, the microscope is compact so that it can be used in confined spaces, such as, for example, vacuum chambers or low-temperature chambers.

In particular, for this application to the construction of an atomic force microscope, it may be advantageous to use two detectors located on and under the beam with a differential detection mode.

In accordance with a second application, an analyzer for analyzing the composition of a fluid comprises at least one microelectromechanical system according to FIG. 1b, which is placed in a microchannel or in a large container. A solid chemical compound, which is capable of selectively reacting with entities of a defined chemical species, is attached to the beam of the system. The solid compound may for example be a gold layer. The fluid is injected into the microchannel or into the container and comes into contact with the compound on the beam. When the fluid contains entities of the species in question, these react with the compound and generate stresses in the latter. These stresses induce a deformation of the beam that is detected and compared with a known reference deformation. The intensity of the deformation depends on the concentration of the entities of the chemical species in the fluid. Several analyzers of this type may be placed in the same microchannel, either to detect variations in the concentration of the entities of any one species along the flow of the fluid or to simultaneously detect the presence of entities of different species. In the latter case, the compounds attached to the beams of the systems are selected according to the various species that are desired to be present in the fluid.

The construction of biodetectors that do not require molecular markers constitutes a third application of a microelectromechanical system according to the invention. Such a biodetector may comprise a microelectromechanical system according to the invention in which the deformable part is designed to be deformed when molecules initially contained in an analyzed fluid are adsorbed in a defined zone of this deformable part. The adsorption of the molecules modifies the surface energy of the deformable part in the adsorption zone and produces a stress. This results in a deformation that is detected by a detector located close to the adsorption zone.

According to a first mode of operation of such a biodetector, the molecules contained in the analyzed fluid are directly adsorbed on the deformable part. For example, this deformable part may comprise a gold layer in the adsorption zone, and the molecules are adsorbed on the gold layer via thiol (—SH₂) end groups carried by the molecules themselves. FIG. 3b illustrates schematically such an operation of a biodetector constructed from a microelectromechanical system according to FIG. 1b or 1c. The beam 1 is covered with the gold layer 13, and the molecules M that are initially free in the analyzed fluid each have a thiol end group shown symbolically by the letter S. As is known, each thiol functional group may form a link between the corresponding molecule and the layer 13 when the molecule is adsorbed on the beam 1.

According to a second mode of operation, the molecules initially contained in the analyzed fluid are adsorbed on the deformable part via chemical functional groups grafted onto the latter. The deformable part of the microelectromechanical system is then surface-modified in the adsorption zone by the chemical functional groups. These chemical functional groups are chosen for selectively attaching certain molecules. Detectors for detecting molecules of different types can therefore be produced by varying only the chemical functional groups that are grafted onto the deformable part of the microelectromechanical system. FIG. 3c corresponds to FIG. 3b for this second mode of operation. The deformable part formed by the beam 1 again includes the gold layer 13. Chemical functional groups 14, for example of the acid type converted to an active ester, which are denoted by —COONHS with reference to the principal atoms of these functional groups, are grafted onto the layer 13 via thiol end groups shown symbolically by the letter S. The functional groups 14 form a permanent molecular layer on the layer 13, which may be self-organized. In this case, the molecules 14 are oriented parallel to one another. The molecules M may thus be attached to the beam 1 with a higher density, thereby allowing a concentration of the molecules present in the fluid to be measured within a more extended concentration range. Such a biodetector is suitable for molecules M of the DNA type that contain an amino (—NH₂) functional group. Attaching such a molecule M to the beam 1 results in a peptide link established between the amino functional group of the molecule and one of the functional groups 14 grafted onto the layer 13.

The construction of a biological analyzer constitutes a fourth application of microelectromechanical systems according to the invention. The principle of operation of such analyzers is based on the variation in a surface tension of a deposit of cells placed on the deformable part of the microsystem. The surface tension generates a deformation of the microsystem, which is detected. In particular, such a biological analyzer may be suitable for carrying out a medical analysis. Given the small size of the analyzer, it can be installed in patient reception areas. Such an organization makes it possible to avoid cellular specimens taken from patients having to be sent to remote laboratories. This results in a reduction in the necessary logistics and in the time needed for the results to be sent back to each patient.

A first advantage of the use of a microelectromechanical system according to the invention in applications for analyzing the composition of a fluid, for biodetection and for biological analysis lies in the absence of a light source for detecting the deformations undergone by the system. This is because such a light source could induce photochemical reactions liable to impair the results obtained.

A second advantage results from the high sensitivity of the stress detector employed. This is because when a detector of limited sensitivity is used in a microelectromechanical system having a deformable part, it is necessary that the deformable part be supple so that the amplitude of the deformations is sufficient. The system then has large time constants that prevent rapid analyses from being carried out. Typically, the phenomena observed using such a system must last longer than 10 minutes in order to be able to be detected, thereby limiting the observation of transient phenomena to those that vary very slowly. Thanks to the sensitivity of the stress detector used in the invention, the deformable part of the system may have a high stiffness, allowing rapid detections compatible with the observation of short transient phenomena. Furthermore, the rapidity of the detections performed using a system according to the invention is compatible with taking a large number of successive measurements. This rapidity is particularly advantageous for medical analysis applications in which a large number of specimens have to be analyzed in succession, corresponding for example to different patients.

In accordance with a fifth application, the microelectromechanical system may constitute an accelerometer. For example when a system having a beam in accordance with FIGS. 1a to 1c is subjected to an acceleration parallel to the direction N, the beam 1 deforms by flexure under the effect of its inertia. The curvature of the beam depends on the value of the acceleration. When the detector is permanently supplied with current via the connections 4a and 4d, the electrical voltage detected between the connections 4b and 4c constitutes a real-time measurement of the acceleration. Such an accelerometer is particularly reliable, simple and inexpensive. It may be easily incorporated into many devices such as, in particular, airbags. The system then allows the airbag to inflate when the acceleration corresponds to a detected voltage above a predefined threshold.

In accordance with a sixth application, the microelectromechanical system may constitute a detector for detecting the deformation of a support. For example, in accordance with FIG. 3d, the system may comprise a beam 1, the two opposed ends 12a and 12b of which are connected to two sides of a fixed part 110. The beam 1 joins two opposed portions of the fixed part, 110a and 110b respectively, forming a bridge over the top of an intermediate portion 110c of the part 110. The part 110 is intended to be attached via its lower face to a support, the deformations of which it is desired to measure. The portion 110c is thinner along the direction N than the portions 110a and 110b so that the part 110 can be deformed with the support, by flexure of the portion 110c. This deformation induces stresses at the ends 12a and 12b of the beam 1 and along the length of the latter, which may be measured by means of a stress detector 10. The detector 10 may be located on one of the ends 12a or 12b, or else between the latter, depending on the position where the stresses are largest. In this way, it is possible in particular to study deformations undergone by part of an airplane, part of a building structure, part of a bridge, a rail or part of a drilling head. It is also possible in this way to detect the deformations of a membrane subjected to variable pressures exerted on either side of the latter and to deduce therefrom a measurement of the difference between the pressures.

The invention may also operate in dynamic mode. The beam 1 is set in vibration at a frequency close to or equal to the frequency of an oscillation eigenmode, using a known method of excitation. This vibration produces a measurable stress that varies periodically as a function of time. Measurement of the amplitude or the phase of the vibrations, or measurement of a variation in their frequency, by means of one or two stress detectors, is useful for many additional applications.

For example, a microelectromechanical system used in dynamic mode may constitute a fluid density analyzer. This is because the damping of the vibrations of the moving part depends on the density of the fluid with which this part is in contact. The denser the fluid, the more rapidly the oscillations are damped. By measuring the amplitude or the phase of the vibrations, it is therefore possible to determine the density of the fluid.

A microelectromechanical system used in dynamic mode may also constitute a mass sensor. The oscillation eigenfrequency of a beam, denoted by f₀, depends on the mass m of this beam according to the known equation f₀²=k/m for a harmonic oscillator, k being a stiffness constant of the beam. Adsorption of molecules or other weighty species on the beam 1 modifies the apparent mass of the latter and changes its eigenfrequency in a manner that can be measured by one or two stress detectors.

A second process for producing a microelectromechanical system according to the invention will now be described with reference to FIGS. 4a-4d, 5 and 6. This second process results in a microelectromechanical system in which the base and shunt portions are integrated into the deformable part, without protruding from the latter (FIG. 6).

A lithographic resist mask M′1 is deposited on an SOI substrate identical to that illustrated in FIG. 2a, which mask covers the upper layer 102 except for an opening corresponding to a portion 104 of the layer 102 (FIG. 4a). The portion 104 is then selectively doped using an ion implantation beam I1. The beam I1 is directed via the opening in the mask M′1 against the upper surface of the layer 102, parallel to the direction N but in the opposite sense thereto. The nature of the ions of the beam I1 and their acceleration voltage are determined in a manner known per se according to the type of doping that it is desired to carry out in the portion 104 and on the thickness of the portion 104. Preferably, this doping is of n-type and the beam I1 is a beam of phosphorus (P) ions. Doping with a first concentration is thus carried out in the portion 104, which becomes conducting. This first concentration may be between 10¹⁴ cm⁻³ and 10²⁰ cm⁻³ when the layer 102 is made of silicon. The portion 104 may have, for example, a thickness of 500 nm.

The mask M′1 is removed and replaced with a lithographic mask M′2 that has an opening O located above part of the portion 104 corresponding to the shunt portion 3 to be produced (FIG. 4b). The mask M′2 covers the upper surface of the layer 102 outside of the shunt portion 3. That part of the portion 104 covered by the mask M′2 corresponds to the base portion 2 of the stress detector.

A second doping is then carried out on the exposed part of the portion 104 by means of a second ion implantation beam I2. This second doping is of the same type as the first, with a higher concentration, for example of the order of 10²¹ cm⁻³. It defines the shunt portion 3. This way of operating, consisting in performing the second doping of the shunt portion 3 within the conducting portion 104 that has received beforehand the first doping, ensures that there is suitable electrical contact between the portions 2 and 3. Furthermore, the continuity of material of the layer 102 between the portions 2 and 3 ensures that no crack will subsequently appear between the portions 2 and 3. The detector obtained will then have a very long lifetime.

A metallic layer 30, for example made of gold, is then deposited on the system (FIG. 4c). In the opening O, part of this layer is in contact with the shunt portion 2.

The mask M′2 is removed so as to leave on the system only that part of the layer 30 which is located on the shunt portion 2.

The substrate 110 is then heated to a temperature high enough for the metal of the residual part of the layer 30 to diffuse into the shunt portion 3 (FIG. 4d). The heating temperature may be, for example, equal to or above 400° C. After this heating, the shunt portion 3 has an electrical behavior of the metallic type. Its electrical conductivity is then very much greater than that of the base portion 2 conferred by only the first doping.

The stress detector is then produced. The deformable part of the microelectromechanical system, which may be in the form of a beam, and electrical connections may then be produced in the manner already described with reference to FIGS. 2g-2i.

The system obtained is shown in FIG. 6. Unlike the system shown in FIG. 1a, the detector 10 is now integrated into the beam 1 without projecting from the upper surface of the latter. In this way, the mechanical properties of the beam 1 are not impaired.

Furthermore, the detector 10 in FIG. 6 is connected only via two electrical connections, with the references 4a and 4b, which simultaneously supply the detector with current and detect the electrical voltage present in the portion 2.

Optionally, these connections, irrespective of their number, may be produced at the same time as the shunt portion 3 without requiring additional specific steps. To do this, the mask M2′ has openings that are shown in the plan view of FIG. 5. Apart from the opening O, the mask M2′ has two additional openings, Oa and Ob, which correspond to the two connections 4a and 4b respectively. The steps described above with reference to FIGS. 4b-4d are carried out simultaneously in that part of the layer 102 corresponding to the portion 3 and in those parts that correspond to the electrical connections. Preferably, the mask M′1 used for the first doping also has openings identical to those Oa and Ob of the mask M′2. The connections 4a and 4b then automatically possess functional electrical contacts with the base portion 2.

Finally, the first electrically conductive material, which constitutes the base portion 2, is preferably selected so that it has a variable electrical conductivity, the anisotropy of which changes when the portion 2 is subjected to a stress. Such an anisotropy, which depends on the stress undergone by the detector 10, contributes to increasing the variation in the lines of current within the base portion 2, in addition to other physical effects. The sensitivity of the detector 10 can be increased in this way.

For example, this first conductive material may be based on substantially single-crystal silicon of cubic structure. The base 2 and shunt 3 portions are then advantageously formed in such a way that they have respective adjoining sides approximately perpendicular to a [110] axis of the silicon of the first conductive material. Such an orientation of the interface between the portions 2 and 3 relative to the crystal axes of the portion 2 maximizes the effect of the anisotropy on the sensitivity of the detector. To do this, the layer 102 of the substrate 110 may initially be made of single-crystal silicon. The substrate 110 can then be rotated parallel to its upper surface, by the start of the fabrication of the microelectromechanical system, so that the longitudinal direction L of the beam 1 to be produced is parallel to the [110] axis of the layer 102, or makes a small angle with this crystallographic axis.

Of course, the invention is not limited to the microelectromechanical systems described in detail with reference to FIGS. 1a-1c. Adaptations of these systems may be carried out depending on the envisioned applications. In particular, the shape, the material and the dimensions of the deformable part and/or those of the stress detector(s), together with the number of electrical connections that connect the base part of each of the detectors, may be modified depending on the specific requirements of each application.

To give an example of such modifications, the stress detector may be oriented, relative to the stresses that are intended to be measured, differently from that illustrated by the figures. The interface between the base portion and the shunt portion may be perpendicular to a direction of the stresses, but, alternatively, it may be oriented parallel to the direction of the stresses or, optionally, may be oriented obliquely to said direction. The orientation of the interface between the base and shunt portions may in particular be chosen so as to obtain a maximum detection sensitivity when the measured stresses have an initially known direction.

1. A microelectromechanical system comprising a deformable part and at least one stress detector firmly joined to said deformable part, each detector comprising:
a base portion and a shunt portion which are electrically conducting and juxtaposed on the deformable part in such a way that the base portion and the shunt portion are in electrical contact with each other along respective adjoining sides of said portions, the shunt portion having an electrical conductivity higher than the conductivity of the base portion; and
a set of electrical connections that are connected to the base portion, away from a contact zone where said base portion is in contact with the shunt portion, and are arranged so that a modification in the distribution of an electrical current in the base and shunt portions may be detected electrically using said connections, in which system respective materials of the base and shunt portions are chosen so that a contact resistance between said portions varies as a function of a deformation of the system.

2. The microelectromechanical system as claimed in claim 1, in which the base portions are integrated into the deformable part without protruding from a surface of said deformable part.

3. The microelectromechanical system as claimed in claim 1, in which the base portion is made of a semiconductor material and the shunt portion is of metallic type.

4. The microelectromechanical system as claimed in claim 1, in which a material of the base portion has a variable electrical conductivity, the anisotropy of which changes when said base portion material is subjected to a stress.

5. The microelectromechanical system as claimed in claim 4, in which the base portion is based on substantially single-crystal silicon of cubic structure, with a axis approximately perpendicular to the respective adjoining sides of said base and shunt portions.

6. The microelectromechanical system as claimed in claim 1, which comprises two stress detectors firmly joined to the deformable part and placed so as to provide a differential measurement of a deformation of the deformable part, for example placed on two opposed faces of said deformable part.

7. The microelectromechanical system as claimed in claim 6, which further includes at least one electronic differential measurement circuit, said circuit being connected to electrical connections, connecting the two detectors respectively, and being integrated into the microelectromechanical system.

8. (canceled)

9. The microelectromechanical system as claimed in claim 7, in which the deformable part comprises a beam.

10. The microelectromechanical system as claimed in claim 9, which further includes a fixed part and in which system the beam has a free end and an end rigidly connected to said fixed part.

11. The microelectromechanical system as claimed in claim 10, in which at least one stress detector is placed close to the end of the beam connected to the fixed part.

12. The microelectromechanical system as claimed in claim 9, in which the electrical connections are arranged on the base portion of each detector in such a way that a flexural deformation of the beam can be detected electrically by means of said connections.

13. The microelectromechanical system as claimed in claim 9, in which the electrical connections are arranged on the base portion of each detector in such a way that deformations of the beam in two different modes can be detected separately be means of said connections.

14. An atomic force microscope comprising a microelectromechanical system as claimed in claim 10, the beam forming part of one arm of said microscope intended to detect variations in at least one structural, physical and/or chemical property of an observed surface, such as variations in height or in friction of said surface.

15. An analyzer for analyzing the composition of a fluid, comprising a microelectromechanical system as claimed in claim 1.

16. A biodetector comprising a microelectromechanical system as claimed in claim 1, in which the deformable part is designed to be deformed when molecules initially contained in an analyzed fluid are adsorbed in a defined zone of said deformable part.

17. (canceled)

18. (canceled)

19. (canceled)

20. A biological analyzer, comprising a microelectromechanical system as claimed in claim 1.

21. (canceled)

22. (canceled)

23. (canceled)

24. (canceled)

25. A sensor for obtaining a parameter of a fluid comprising a microelectromechanical system as claimed in claim 1 said parameter being selected from a density, a temperature or a flow rate of said fluid, or a mass deposited by said fluid on the moving part, said sensor being designed to detect a variation in an amplitude or frequency of vibration of the moving part caused by a variation in said parameter of the fluid.

26-33. (canceled)
